(19) United States
(12) Patent Application Publication
(10) Pub. No.: US 2025/0028015 A1
(43) Pub. Date: Jan. 23, 2025

(54) INTRAVOXEL TWO COMPARTMENT MRI PHANTOM

(71) Applicant: CaliberMRI, Inc., Boulder, CO (US)

(72) Inventor: William Hollander, Boulder, CO (US)

(21) Appl. No.: 18/713,950

(22) PCT Filed: Nov. 28, 2022
(86) PCT No.: PCT/US2022/051090

§ 371 Date: May 28, 2024

Related U.S. Application Data
(60) Provisional application No. 63/283,448, filed on Nov. 27, 2021.

(51) Int. Cl. G01R 33/58

(52) U.S. Cl. CPC G01R 33/58

(57) ABSTRACT

An MRI phantom includes a first chamber containing a first fluid having a first set of mimic characteristics, such as T2 mimic characteristics, and a second chamber containing a second fluid having a second set of mimic characteristics, such as T2 mimic characteristics. The MRI phantom further includes a physical separator defining at least two interleaved regions of the first and second chambers, at least one of the at least two interleaved slits allowing the first fluid to flow therein and at least another of the at least two interleaved regions allowing the second fluid to flow therein without the first and second fluids mixing together.

### CROSS-REFERENCE TO RELATED APPLICATIONS

This application claims the benefit of U.S. provisional application No. 63/283,448, filed 27 Nov. 2021, which is hereby incorporated by reference as though fully set forth herein.

### BACKGROUND

### a. Field

The instant disclosure relates to a phantom having one or more known characteristics for use in calibrating an MRI instrument or in calibration and validation of MRI measurement methodologies including software tools.

### b. Background

Calibration of MRI devices and validation of MRI measurement methodologies can be accomplished via the use of a “phantom” that has one or more known characteristics. The phantom is inserted into the MRI device, a calibration sequence is initiated and performed, and resulting measurements allow for proper calibration of measurements made using the calibrated MRI device. The inventors of the present invention have recognized that an advanced phantom device that allows for easy MRI instrument calibration and MRI methodology validation would be useful. It would therefore be beneficial to provide such a device.

### BRIEF SUMMARY

Provided here is a novel MRI phantom, referred to as the Intravoxel Two Compartment (IV2C) MRI Phantom. The IV2C MRI phantom as described in this disclosure allows two different MRI mimic compositions, such as discrete T2 mimics in a luminal water phantom, to come close to each other, while not actually mixing. As an example of the IV2C MRI phantom, the close proximity of these discrete fluids, and the particular positional relationship between the fluids is such that their magnetic resonance characteristics would appear to be blended from the perspective of the MRI scanner. This is accomplished by separating the two fluids with a relatively thin barrier or separator, where the thinness of that barrier is judged relative to the voxel (volumetric pixel) dimensions of the MRI scan. The end result is that both fluids are sampled, in known proportions to each other, in a given voxel, allowing the study of different fluids in close proximity to each other, potentially emulating discrete biological formations.

Embodiments a phantom may include the following:

An MRI phantom of this invention can have a first chamber containing a first fluid having a first set of tissue mimic characteristics, a second chamber containing a second fluid having a second set of tissue mimic characteristics; wherein the first and second chambers each have one or more protruding leaves; and wherein the protruding leaves of the first and second chambers are interleaved within the phantom. The interleaving of the protruding leaves may be a dovetail arrangement. The first chamber can hold a first fluid different from the second fluid in the second chamber.

The phantom above, wherein the protruding leaves may be formed by a separator disposed between the first chamber and the second chamber, the separator defining at least one protruding leaf in fluid communication with the first chamber and at least one protruding leaf in fluid communication with the second chamber, wherein the protruding leaves are interleaved. The separator may be integrated with the walls of the first chamber, or may be integrated with the walls of the second chamber, or may be integrated with the walls of both the first and second chambers.

The phantom above, wherein the protruding leaves can be interleaved so that alternating leaves are in fluid communication with different chambers.

The phantom above, wherein the protruding leaves can be interleaved so that a repeating pattern of leaves are in fluid communication with different chambers. The dovetailing of the protruding leaves may be in pairs, triplets, or any other repeating pattern.

The phantom above, wherein the protruding leaves may be planar, convex or concave, saddle-shaped, or have elongated geometric or irregular shape. The protruding leaves can be any shape that can be stacked together so that at one or more points within the phantom the first fluid can be separated from the second fluid by only the thickness of a chamber wall of a protruding leaf.

The phantom above, wherein the interleaved protruding leaves contain the first fluid in the first chamber in close proximity to the second fluid in the second chamber.

The phantom above, wherein at least two of the protruding leaves are within a single MRI voxel of interest.

The phantom above, wherein the protruding leaves have a thickness less than the thickness of an MRI voxel of interest.

The phantom above, wherein the protruding leaves have a thickness less than two millimeters.

The phantom above, wherein the interleaved protruding leaves contain the first fluid in the first chamber in proximity to the second fluid in the second chamber, wherein the first chamber and the second chamber are not in fluid communication.

The phantom above, wherein the separator is integrated with the first and second chambers.

The phantom above, wherein at least one of the tissue mimic characteristics of the first set of tissue mimic characteristics is a relaxation time of the first fluid.

The phantom above, wherein at least one of the tissue mimic characteristics of the second set of tissue mimic characteristics is a relaxation time of the second fluid.

The phantom above, wherein at least one of the tissue mimic characteristics is a relaxation time T2 of a luminal water imaging method.

The phantom above, wherein within the interleaved protruding leaves the MRI signal of the first fluid in the first chamber has a predetermined ratio to the MRI signal of the second fluid in the second chamber.

The phantom above, comprising a top layer positioned above the separator, a middle layer for supporting the separator, and a lower layer positioned below the separator.

The phantom above, wherein the top layer, the middle layer and the separator define the first chamber, and wherein the bottom layer, the middle layer and the separator define the second chamber.

The phantom above, wherein the components of the phantom are secured together with fasteners making fluid-tight seals which contain the first fluid in the first chamber and the second fluid in the second chamber. The fluids may be retained in the chambers for a long period of time, for example months or years, without leakage.

The phantom above, comprising a first port in the first chamber for accessing the first chamber, and a second port in the second chamber for accessing the first chamber.

A method for calibrating an MRI instrument or validating an MRI scanning method, by providing an MRI phantom above, wherein the phantom is filled with a first fluid in the first chamber and a second fluid in the second chamber; and acquiring an MRI scan of at least one voxel of interest in the phantom. The voxel of interest may comprise a portion of the first fluid and a portion of the second fluid.

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1 is perspective view of a phantom constructed in accordance with an embodiment of this disclosure;

FIG. 2 is an exploded view of the phantom of FIG. 1;

FIG. 3 is a top plan view of the phantom of FIG. 1;

FIG. 4 is a bottom plan view of the phantom of FIG. 1;

FIG. 5 is a partially transparent perspective view of the phantom of FIG. 1;

FIG. 6 is a cross-sectional view taken along line 6-6 of FIG. 1.

FIG. 7 is a a cross-sectional view of FIG. 1 taken along line 7-7; and

FIG. 8 is a perspective view of a phantom device with an MRI instrument useful such as for calibration of the MRI instrument and/or validation an MRI measure methodology.

### DETAILED DESCRIPTION

The following description of the invention is provided as an enabling teaching of the invention in its best, currently known embodiment. To this end, those skilled in the relevant art will recognize and appreciate that many changes can be made to the various aspects of the invention described herein, while still obtaining the beneficial results of the present invention. It will also be apparent that some of the desired benefits of the present invention can be obtained by selecting some of the features of the present invention without utilizing other features. Accordingly, those who work in the art will recognize that many modifications and adaptations to the present invention are possible and can even be desirable in certain circumstances and are a part of the present invention. Thus, the following description is provided as illustrative of the principles of the present invention and not in limitation thereof.

As used throughout, the singular forms “a,” “an” and “the” include plural referents unless the context clearly dictates otherwise. Thus, for example, reference to “a” component can include two or more such components unless the context indicates otherwise. Also, the words “proximal” and “distal” are used to describe items or portions of items that are situated closer to and away from, respectively, a user or operator such as a surgeon. Thus, for example, the tip or free end of a device may be referred to as the distal end, whereas the generally opposing end or handle may be referred to as the proximal end.

All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “optional” or “optionally” mean that the subsequently described event or circumstance may or may not occur, and that the description includes instances where said event or circumstance occurs and instances where it does not.

The term “substantially” as used herein may be applied to modify any quantitative representation which could permissibly vary without resulting in a change in the basic function to which it is related.

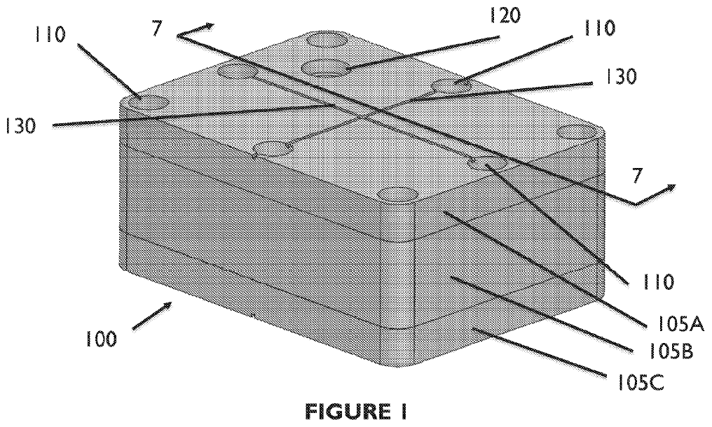

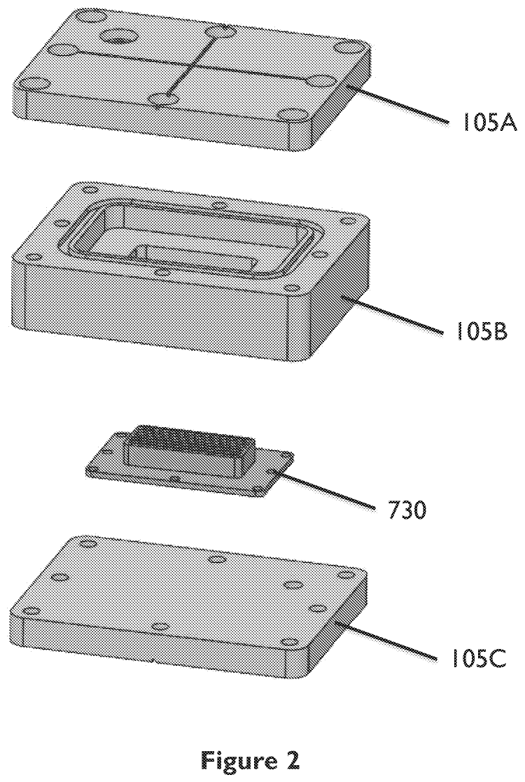

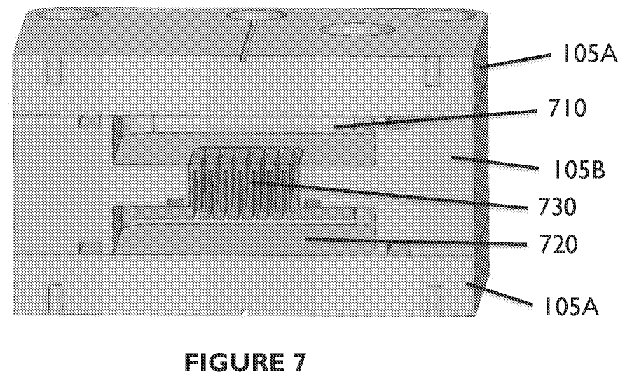

Referring first to FIGS. 1 and 2, a preferred MRI phantom 100 constructed in accordance with a first embodiment may comprise a top layer 105A, a middle layer 105B and a bottom layer 105C secured together, such as by one or more bolts (not shown) secured in through holes 110. When secure, these bolts provide a liquid-tight seal between the various layers 105A, 105B and 105C. Phantom 100 further may comprise a first port 120 for introducing a first liquid into a top chamber 710 (see FIG. 7, a cross-sectional view of FIG. 1 taken along line 7-7), and slits 130. While not shown in FIG. 1 (see FIG. 2), bottom layer 105C similarly includes a second port 120A to allow for introduction of a second liquid into a bottom chamber 720 (see FIG. 7). Finally, referring once again to FIG. 7, a virtually intermingling diaphragm 730 is positioned between top chamber 610 and bottom chamber 620.

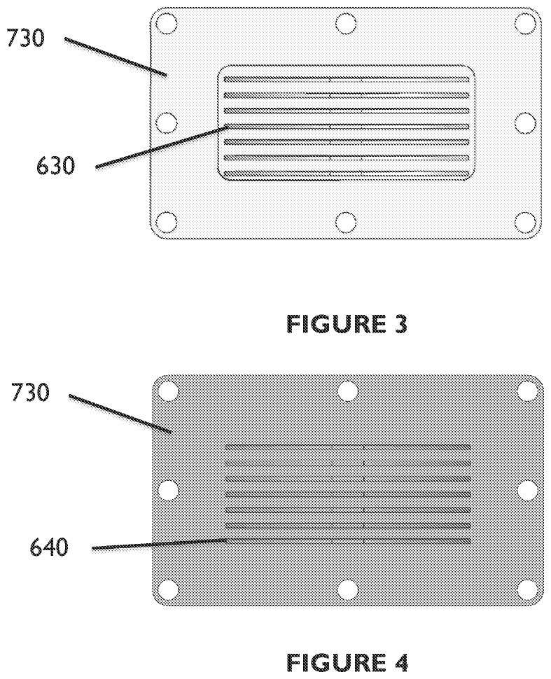

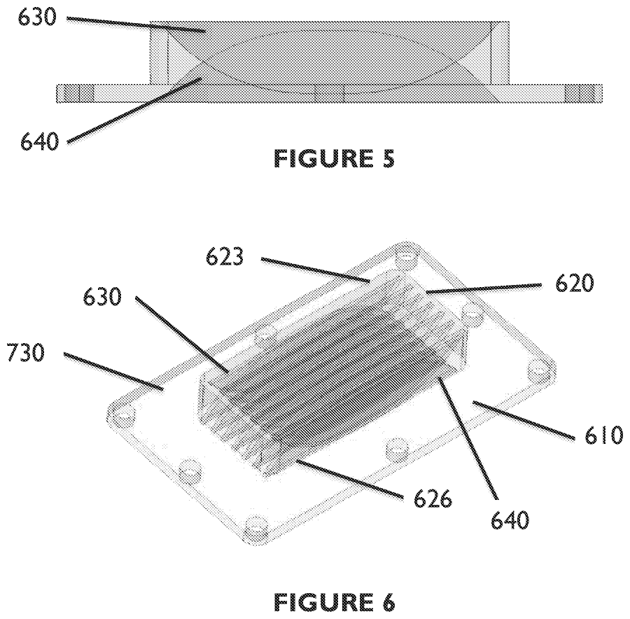

Virtually intermingling diaphragm 730 is shown in greater detail in FIG. 3-6. As is first shown in FIG. 6, diaphragm 730 comprises a raised block portion 620 protruding upward from a base plate portion 610. One or more grooves may be provided on either side of raised block portion 620, preferably resulting in an overlapping pattern. As is shown in FIG. 6, a first set of thin deep grooves 630 may be provided from a upper surface 623 of raised block portion 620, and a second set of thin deep grooves 640 may be provided from a lower surface 626 of raised block portion 620. Grooves 630 and 640 are further shown interleaving in FIG. 5, and in plan view in FIGS. 3 and 4. In a particular embodiment, virtual intermingling diaphragm (or manifold) 730 may be constructed by one or more slitting saws cutting a block of plastic, from two sides. Alternatively, diaphragm 730 may be printed via a 3D printer, or formed via other molding or casting techniques. Thus, thin deep grooves may be created in the plastic or other appropriate material, with the open side of the groove alternating sides of the material, allowing one set of slits to be filled with one solution from one chamber via the first port 120, while the other set of slits may be filled with a different solution from another chamber via the second port 120A. The materials that may be used in the construction of the phantom encompass any material that is substantially invisible to MRI, machinable, stable, stiff, and having low water permeability. Examples of MR-compatible plastics or polymeric materials include acrylonitrile butadiene styrene, polymethyl methacrylate, polylactic acid, polycarbonate, polyethylene, polystyrene, polyvinyl chloride, or polytetrafluoroethylene.

A virtual intermingling region of interest (interleaved grooves) results in unique characteristics of MRI phantom 100. The interleaved grooves allow fluid from one side of the phantom (chamber 710) to come into close proximity to fluid from the other side of the phantom (chamber 720), in a repeating pattern, such that both fluids appear in a single voxel, in a known proportion to each other (based of the width of the slits relative to each other). In this embodiment, the phantom allows for maintaining access for in situ measurements of the individual mimic solutions, such as individual T2 mimic solutions by virtue of having discrete T2 (long) and T2 (short) tanks for the LWI example. This allows simultaneous measurement of the discrete solution characteristics and the integrated T2 within multiple imaging voxels.

The result of the structure of the IV2C MRI Phantom is to create a labyrinthine boundary between two different solutions with different magnetic resonance properties (e.g. different T2 times), so that a single voxel or a series of voxels includes both fluids, yet the two fluids are not allowed to mix. In this way, the geometry of the labyrinthine boundary can be altered to allow for different proportions of the two solutions in a given voxel, so that the constituent make-up of the two voxel components can be varied. This allows for study of how different unmixed fluids, sampled in different proportions to each other, appear to a MR scan, when combined in the same sample element (voxel). Ultimately, this provides a basis for calibration and optimization of the IV2C MRI imaging technique (LWI as example).

The walls separating the interleaved volumes may comprise any thickness. In one implementation, a thickness of 0.1-1.0 mm is utilized, for example, 0.3 or 0.5 mm. The interleaved regions of the device may be of any size, for example, in the range of 1-5 mm, for example, 2 or 3 mm in length. The volumes defined by the interleaved walls may comprise any thickness, for example, in the range of 0.1 to 1.0 mm, for example, 0.5 or 0.7 mm in width.

In an embodiment of a phantom, fluids may comprise different MRI tissue mimic characteristics, such as diffusion or relaxation properties (e.g., T2 mimic characteristics). In a particular embodiment, these fluids may comprise different T2 mimic characteristics, such as a T2(long)=approximately 1 sec, and a T2(short)=approximately 50-60 msec. Other MR parameter sets or characteristics, such as diffusion and/or T1 characteristics, may be used. The phantom can further be configured to enable response from each mimic within a voxel, such as a 2 mm per side voxel. The total phantom may also fill at least 2×2×2 voxels in an embodiment. The phantom can be constructed to allow and ensure accurate, repeatable placement in scanner. In particular embodiments, while fractional response may be considered, relative location may be considered non-critical.

Thus, the materials selection, assembly method and fluid management employed in a phantom can be distinctive in that they provide reliable, accurate T2 mimics with very long-term stability, ultra-low leak rates and compatibility with MR scanner systems requirements. Examples of materials include, but are not limited to ionic aqueous solutions, oils, gels, viscous polymers, water-soluble polymers, purified bodily fluids. In one aspect, a T2 material may include manganese chloride (mncl₂), a T1 material may include nickel chloride (nicl₂), and a Polyvinylpyrrolidone (PVP) for apparent diffusion coefficient (ADC). Other examples include, but are not limited to, agar and/or gadolinium.

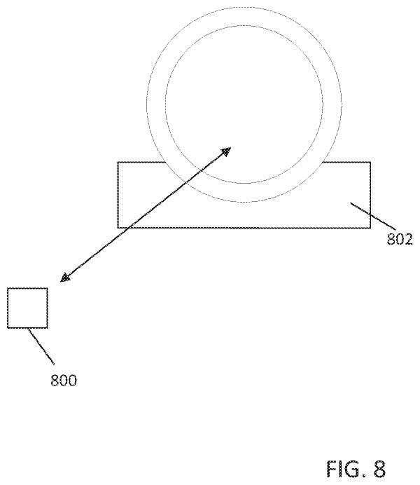

FIG. 8 is a perspective view of a phantom 800 in use in an MRI instrument 802, such as for calibration. In this example, a phantom 800 is placed in an imaging portion of the MRI instrument 802. One or more images of the phantom may be obtained via the MRI instrument.

1. An MRI phantom, comprising:
a first chamber containing a first fluid having a first set of tissue mimic characteristics;
a second chamber containing a second fluid having a second set of tissue mimic characteristics;
wherein the first and second chambers each have one or more protruding leaves; and wherein the protruding leaves of the first and second chambers are interleaved within the phantom.

2. The phantom of claim 1, wherein the protruding leaves are formed by a separator disposed between the first chamber and the second chamber, the separator defining at least one protruding leaf in fluid communication with the first chamber and at least one protruding leaf in fluid communication with the second chamber, wherein the protruding leaves are interleaved.

3. The phantom of claim 1, wherein the protruding leaves are interleaved so that alternating leaves are in fluid communication with different chambers.

4. The phantom of claim 1, wherein the protruding leaves are interleaved so that a repeating pattern of leaves are in fluid communication with different chambers.

5. The phantom of claim 1, wherein the protruding leaves are planar, convex or concave, saddle-shaped, or have elongated geometric or irregular shape.

6. The phantom of claim 1, wherein the interleaved leaves contain the first fluid in the first chamber in close proximity to the second fluid in the second chamber.

7. The phantom of claim 1, wherein at least two of the protruding leaves are within a single MRI voxel of interest.

8. The phantom of claim 1, wherein the protruding leaves have a thickness less than the thickness of an MRI voxel of interest.

9. The phantom of claim 1, wherein the protruding leaves have a thickness less than two millimeters.

10. The phantom of claim 1, wherein the interleaved protruding leaves contain the first fluid in the first chamber in proximity to the second fluid in the second chamber, wherein the first chamber and the second chamber are not in fluid communication.

11. The phantom of claim 2, wherein the separator is integrated with the first and second chambers.

12. The phantom of claim 1, wherein at least one of the tissue mimic characteristics of the first set of tissue mimic characteristics is a relaxation time of the first fluid.

13. The phantom of claim 1, wherein at least one of the tissue mimic characteristics of the second set of tissue mimic characteristics is a relaxation time of the second fluid.

14. The phantom of claim 1, wherein at least one of the tissue mimic characteristics is a relaxation time T2 of a luminal water imaging method.

15. The phantom of claim 1, wherein within the interleaved protruding leaves the MRI signal of the first fluid in the first chamber has a predetermined ratio to the MRI signal of the second fluid in the second chamber.

16. The phantom of claim 2, comprising:
a top layer positioned above the separator,
a middle layer for supporting the separator, and
a lower layer positioned below the separator.

17. The phantom of claim 16, wherein the top layer, the middle layer and the separator define the first chamber, and wherein the bottom layer, the middle layer and the separator define the second chamber.

18. The phantom of claim 2, wherein the components of the phantom are secured together with fasteners making fluid-tight seals which contain the first fluid in the first chamber and the second fluid in the second chamber.

19. The phantom of claim 1, comprising a first port in the first chamber for accessing the first chamber, and a second port in the second chamber for accessing the first chamber.

20. A method for calibrating an MRI instrument or validating an MRI scanning method, the method comprising:
providing an MRI phantom according to any one of claims 1-19, wherein the phantom is filled with a first fluid in the first chamber and a second fluid in the second chamber, and
acquiring an MRI scan of at least one voxel of interest in the phantom.
